(19) United States
(12) Patent Application Publication
(10) Pub. No.: US 2017/0143781 A1
(43) Pub. Date: May 25, 2017

(54) PAIN RELIEF COMPOSITION

(71) Applicant: Leslie Ann Yarborough, Danville, CA (US)

(72) Inventor: Leslie Ann Yarborough, Danville, CA (US)

(21) Appl. No.: 14/950,407

(22) Filed: Nov. 24, 2015

(51) Int. Cl. A61K 36/185; A61K 47/46; A61K 35/10; A61K 31/202; A61K 31/165

(52) U.S. Cl. CPC A61K 36/185; A61K 31/202; A61K 31/165; A61K 35/10; A61K 47/46

(57) ABSTRACT

Natural pain relief and muscle recovery compositions having a wide variety of topical applications are provided. In certain embodiments, the pain relief composition includes moringa leaf, moringa oil, a gamma linolenic acid (GLA) source, and a capsaicin source.

### TECHNICAL FIELD

Example embodiments relate generally to natural pain relief and muscle recovery compositions.

### BACKGROUND

Recently, there has been an increased interest and focus on improving health, both through nutrition and exercise, resulting in more individuals getting involved in intense workouts including, but not limited to CrossFit®, barre, cycling, Pilates, TRX®, running clubs, high intensity interval training and/or the like. As such, there has also recently been an increased interest in and purchasing of topical pain relief treatments in order to relieve sore muscles. However, many of the currently available pain relief treatments include components such as methyl salicylate or camphor, which can be toxic in large doses. Some attempts have been made to produce natural pain relief treatments, but these treatments are not typically sufficiently effective at relieving muscle soreness. Similarly, there has been an increased interest in topical muscle recovery treatments to encourage quick and healthy muscle recovery following strenuous exercise.

Therefore there remains a need in the art for effective, natural pain relief and muscle recovery compositions for topical applications including, but not limited to, relieving sore muscles.

### BRIEF SUMMARY

One or more example embodiments may address one or more of the aforementioned problems. Certain example embodiments provide pain relief compositions having a wide variety of topical applications. In accordance with certain embodiments, the pain relief composition may comprise moringa leaf, moringa oil, a gamma linolenic acid (GLA) source, and a capsaicin source.

### DETAILED DESCRIPTION

Some example embodiments now will be described more fully hereinafter with reference to the accompanying drawings, in which some, but not all embodiments of the inventions are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

An example embodiment includes a pain relief composition. In accordance with certain embodiments, the pain relief composition may comprise moringa leaf, moringa oil, a gamma linolenic acid (GLA) source, and a capsaicin source. In some embodiments, for example, the pain relief composition may also have muscle recovery applications.

The term “cream”, as used herein, may comprise a fluid mixture having a thick consistency. In certain embodiments, for example, a cream may comprise a semisolid dosage form being either an emulsion of oil and water with approximately equal proportions of oil and water or an aqueous microcrystalline dispersion of a long-chain fatty acid or alcohol. Creams may be designed to be used topically and, as such, may be used as a method of applying medication to the surface of the body. For example, creams may be designed to penetrate the stratum corneum outer layer of the skin wall. Creams may exhibit higher viscosity than lotions.

The term “lotion”, as used herein, may comprise a thick, smooth liquid emulsion preparation designed to be applied to the skin for medicinal or cosmetic purposes. Lotions may be emulsions of oil and water or aqueous microcrystalline dispersions of a long-chain fatty acid or alcohol. In addition, lotions may be low- to heavy-viscosity topical preparations, although lotions may exhibit lower viscosity than creams.

The term “oil”, as used herein, may comprise a slippery or viscous liquid or liquifiable substance that is substantially immiscible in water, including any such substance that would find value in a cosmetic preparation. Natural oils may include vegetable oils that consist of aethereal salts of glycerin with a large number of organic acids such as stearic acid, oleic acid, and palmitic acid forming stearin, olein and palmitin, respectively. Stearin and palmitin prevail in the solid oils and fats, while olein is dominant in the liquid oils. Oils penetrate the skin to nourish, revitalize, and soften the skin.

In some example embodiments, a pain relief composition having a wide variety of topical applications is provided. For example, pain relief compositions may be used for relieving sore muscles resulting from extensive physical activity. In general, pain relief compositions according to certain example embodiments may include moringa leaf, moringa oil, a gamma linolenic acid (GLA) source, and a capsaicin source. In certain embodiments, for instance, the pain relief composition may comprise a cream, a lotion, or an oil.

In accordance with an example embodiment and as mentioned above, the pain relief composition may comprise moringa leaf and moringa oil. In this regard, for example, the moringa leaf and the moringa oil may provide at least one of pain relief, anti-inflammatory properties, antiseptic properties, or any combination thereof to the pain relief composition. According to certain embodiments, for instance, the pain relief composition may comprise from about 1 wt. % to about 40 wt. % moringa leaf. In other embodiments, for example, the pain relief composition may comprise from about 2 wt. % to about 20 wt. % moringa leaf. In further embodiments, for instance, the pain relief composition may comprise from about 5 wt. % to about 7 wt. % moringa leaf. In some embodiments, for example, the pain relief composition may comprise about 6 wt. % moringa leaf. For instance, in one embodiment, the pain relief composition may comprise 6.18 wt. % moringa leaf. As such, in certain embodiments, the pain relief composition may comprise moringa leaf at a weight percent from at least about any of the following: 1, 2, 3, 4, 5, and 6 wt. % and/or at most about 40, 30, 20, 15, 10, 7, and 6 wt. % (e.g., about 2-10 wt. % moringa leaf, about 5-20 wt. % moringa leaf, etc.).

In accordance with an example embodiment, the pain relief composition may comprise from about 1 wt. % to about 40 wt. % moringa oil. In other embodiments, for instance, the pain relief composition may comprise from about 2 wt. % to about 20 wt. % moringa oil. In further embodiments, for example, the pain relief composition may comprise from about 5 wt. % to about 7 wt. % moringa oil. In some embodiments, for instance, the pain relief composition may comprise about 6 wt. % moringa oil. For example, in one embodiment, the pain relief composition may comprise 6.18 wt. % moringa oil. As such, in certain embodiments, the pain relief composition may comprise moringa oil at a weight percent from at least about any of the following: 1, 2, 3, 4, 5, and 6 wt. % and/or at most about 40, 30, 20, 15, 10, 7, and 6 wt. % (e.g., about 2-10 wt. % moringa oil, about 5-20 wt. % moringa oil, etc.).

In accordance with an example embodiment, the pain relief composition may comprise a GLA source. In this regard, for example, the GLA source may provide at least one of anti-inflammatory properties, vasodilation, or any combination thereof. In some embodiments, for instance, the GLA source may comprise a natural GLA source. In further embodiments, for example, the GLA source may comprise at least one of evening primrose essential oil, borage seed oil, black currant seed oil, hemp seed oil, or any combination thereof. In certain embodiments, for example, the GLA source may comprise evening primrose essential oil.

According to an example embodiment, for instance, the pain relief composition may comprise from about 0.5 wt. % to about 30 wt. % of the GLA source. In other embodiments, for example, the pain relief composition may comprise from about 1 wt. % to about 10 wt. % of the GLA source. In further embodiments, for instance, the pain relief composition may comprise from about 2 wt. % to about 4 wt. % of the GLA source. In some embodiments, for example, the pain relief composition may comprise about 3 wt. % of the GLA source. For instance, in one embodiment, the pain relief composition may comprise 3.09 wt. % of the GLA source. As such, in certain embodiments, the pain relief composition may comprise the GLA source at a weight percent from at least about any of the following: 0.5, 1, 1.5, 2, 2.5, and 3 wt. % and/or at most about 30, 20, 10, 5, 4, and 3 wt. % (e.g., about 2-5 wt. %, about 1-20 wt. %, etc.).

In accordance with an example embodiment, the pain relief composition may comprise a capsaicin source. In this regard, for example, the capsaicin source may provide at least one of pain relief, warming properties, or any combination thereof to the pain relief composition. In some embodiments, for instance, the capsaicin source may comprise a natural capsaicin source. In further embodiments, for example, the capsaicin source may comprise at least one of cayenne essential oil, black pepper essential oil, or any combination thereof. In certain embodiments, for instance, the capsaicin source may comprise cayenne essential oil.

According to an example embodiment, for instance, the pain relief composition may comprise from about 0.025 wt. % to about 20 wt. % of the capsaicin source. In other embodiments, for example, the pain relief composition may comprise from about 0.25 wt. % to about 5 wt. % of the capsaicin source. In further embodiments, for instance, the pain relief composition may comprise from about 0.5 wt. % to about 1 wt. % of the capsaicin source. In some embodiments, for example, the pain relief composition may comprise from about 0.6 wt. % to about 0.8 wt. % of the capsaicin source. In further embodiments, for instance, the pain relief composition may comprise about 0.75 wt. % of the capsaicin source. For example, in one embodiment, the pain relief composition may comprise 0.77 wt. % of the capsaicin source. As such, in certain embodiments, the pain relief composition may comprise the capsaicin source at a weight percent from at least about any of the following: 0.025, 0.1, 0.25, 0.5, 0.6, 0.7, and 0.75 wt. % and/or at most about 20, 10, 5, 4, 3, 2, 1, 0.9, 0.8, and 0.75 wt. % (e.g., about 0.6-0.9 wt. %, about 0.25-0.8 wt. %, etc.).

In accordance with an example embodiment, the pain relief composition may further comprise fulvic acid. In this regard, for example, fulvic acid may improve the opening of membranes upon application of the pain relief composition on the skin to allow for improved absorption. According to certain embodiments, for instance, the pain relief composition may comprise from about 0.5 wt. % to about 30 wt. % fulvic acid. In other embodiments, for example, the pain relief composition may comprise from about 1 wt. % to about 10 wt. % fulvic acid. In further embodiments, for instance, the pain relief composition may comprise from about 2 wt. % to about 4 wt. % fulvic acid. In some embodiments, for example, the pain relief composition may comprise about 3 wt. % fulvic acid. For instance, in one embodiment, the pain relief composition may comprise 3.09 wt. % fulvic acid. As such, in certain embodiments, the pain relief composition may comprise fulvic acid at a weight percent from at least about any of the following: 0.5, 1, 1.5, 2, 2.5, and 3 wt. % and/or at most about 30, 20, 10, 5, 4, and 3 wt. % (e.g., about 0.5-4 wt. %, about 3-10 wt. %, etc.).

In accordance with an example embodiment, the pain relief composition may further comprise geranium essential oil. In this regard, for example, the geranium essential oil may provide a natural preservative to the pain relief composition to improve the shelf life of the pain relief composition. According to certain embodiments, for instance, the pain relief composition may comprise from about 0.025 wt. % to about 10 wt. % geranium essential oil. In other embodiments, for example, the pain relief composition may comprise from about 0.05 wt. % to about 1 wt. % geranium essential oil. In further embodiments, for instance, the pain relief composition may comprise from about 0.1 wt. % to about 0.5 wt. % geranium essential oil. In some embodiments, for example, the pain relief composition may comprise from about 0.2 wt. % to about 0.4 wt. % geranium essential oil. For instance, in one embodiment, the pain relief composition may comprise 0.39 wt. % geranium essential oil. As such, in certain embodiments, the pain relief composition may comprise geranium essential oil at a weight percent from at least about any of the following: 0.025, 0.05, 0.075, 0.1, 0.15, and 0.2 wt. % and/or at most about 10, 5, 1, 0.9, 0.8, 0.7, 0.6, 0.5, and 0.4 wt. % (e.g., about 0.075-0.4 wt. %, about 0.2-0.8 wt. %, etc.).

In accordance with an example embodiment, the pain relief composition may further comprise a plurality of natural inactive ingredients. In some embodiments, for instance, the pain relief composition may comprise at least one of cocoa butter, shea butter, coconut oil, olive oil, avocado oil, or any combination thereof. As will be understood by one of ordinary skill in the art, the types and amounts of the natural inactive ingredients in the pain relief composition will depend on whether the pain relief composition comprises a cream, a lotion, or an oil. By way of example only, a cream or a lotion may include cocoa butter, shea butter, and coconut oil, but an oil may include cocoa butter and shea butter and replace coconut oil with either olive oil or avocado oil. Moreover, an oil, for instance, may include more geranium essential oil than a cream or a lotion. In this regard, the consistency of the pain relief composition may be controlled by changing the types and amounts of natural inactive ingredients.

According to an example embodiment, the pain relief composition may comprise from about 1 wt. % to about 60 wt. % cocoa butter. In other embodiments, for example, the pain relief composition may comprise from about 2 wt. % to about 40 wt. % cocoa butter. In further embodiments, for instance, the pain relief composition may comprise from about 5 wt. % to about 25 wt. % cocoa butter. In some embodiments, for example, the pain relief composition may comprise from about 8 wt. % to about 10 wt. % cocoa butter. In certain embodiments, for instance, the pain relief composition may comprise about 9 wt. % cocoa butter. For example, in one embodiment, the pain relief composition may comprise 9.27 wt. % cocoa butter. As such, in certain embodiments, the pain relief composition may comprise cocoa butter at a weight percent from at least about any of the following: 1, 2, 3, 4, 5, 6, 7, 8, and 9 wt. % and/or at most about 60, 50, 40, 30, 25, 20, 15, and 10 wt. % (e.g., about 4-25 wt. %, about 5-10 wt. %, etc.).

In accordance with an example embodiment, the pain relief composition may comprise from about 20 wt. % to about 80 wt. % shea butter. In other embodiments, for example, the pain relief composition may comprise from about 30 wt. % to about 70 wt. % shea butter. In further embodiments, for instance, the pain relief composition may comprise from about 40 wt. % to about 60 wt. % shea butter. In some embodiments, for example, the pain relief composition may comprise from about 54 wt. % to about 56 wt. % shea butter. In certain embodiments, for instance, the pain relief composition may comprise about 55 wt. % shea butter. For example, in one embodiment, the pain relief composition may comprise 55.6 wt. % shea butter. As such, in certain embodiments, the pain relief composition may comprise shea butter at a weight percent from at least about any of the following: 20, 25, 30, 35, 40, 45, 50, 54, and 55 wt. % and/or at most about 80, 75, 70, 65, 60, 58, and 56 wt. % (e.g., about 50-60 wt. %, about 40-65 wt. %, etc.).

In accordance with an example embodiment, the pain relief composition may comprise from about 1 wt. % to about 60 wt. % coconut oil. In other embodiments, for example, the pain relief composition may comprise from about 2 wt. % to about 40 wt. % coconut oil. In further embodiments, for instance, the pain relief composition may comprise from about 5 wt. % to about 25 wt. % coconut oil. In some embodiments, for example, the pain relief composition may comprise from about 8 wt. % to about 10 wt. % coconut oil. In certain embodiments, for instance, the pain relief composition may comprise about 10 wt. % coconut oil. For example, in one embodiment, the pain relief composition may comprise 9.27 wt. % coconut oil. As such, in certain embodiments, the pain relief composition may comprise coconut oil at a weight percent from at least about any of the following: 1, 2, 3, 4, 5, 6, 7, 8, 9, and 10 wt. % and/or at most about 60, 50, 40, 30, 25, 20, 15, and 10 wt. % (e.g., about 4-25 wt. %, about 5-10 wt. %, etc.).

In accordance with an example embodiment, the pain relief composition may further comprise a fragrance. In some embodiments, for instance, the fragrance may comprise at least one essential oil. The at least one essential oil may be any essential oil understood by one of ordinary skill in the art to be acceptable for topical application including, but not limited to, at least one of amber essential oil, star anise essential oil, basil essential oil, bay leaf essential oil, benzoin essential oil, bergamot essential oil, cajeput essential oil, calendula essential oil, carrot seed essential oil, cassia essential oil, cedar essential oil, chamomile essential oil, Clary sage essential oil, coriander essential oil, cypress essential oil, sweet fennel essential oil, frankincense essential oil, grapefruit essential oil, jasmine essential oil, juniper berry essential oil, lavender essential oil, lemongrass essential oil, sweet marjoram essential oil, myrrh essential oil, neroli essential oil, orange blossom essential oil, palmarosa essential oil, peppermint essential oil, petitgrain essential oil, rose essential oil, rosemary essential oil, spearmint essential oil, tangerine essential oil, tea tree essential oil, thyme essential oil, vanilla essential oil, ylang ylang essential oil, or any combination thereof. In certain embodiments, for example, the at least one essential oil may comprise lavender essential oil, grapefruit essential oil, vanilla essential oil, amber essential oil, cedar essential oil, orange blossom essential oil, or any combination thereof. In further embodiments, for example, the fragrance may comprise at least one natural scented oil. In such embodiments, for instance, the at least one natural scented oil may comprise any natural scented oil suitable as a fragrance for the pain relief composition as understood by one of ordinary skill in the art.

According to certain embodiments, for instance, the pain relief composition may comprise from about 0.025 wt. % to about 40 wt. % fragrance. In other embodiments, for example, the pain relief composition may comprise from about 0.1 wt. % to about 20 wt. % fragrance. In further embodiments, for instance, the pain relief composition may comprise from about 1 wt. % to about 10 wt. % fragrance. In some embodiments, for example, the pain relief composition may comprise from about 2 wt. % to about 4 wt. % fragrance. By way of example only, in one embodiment, for instance, the fragrance may comprise lavender essential oil and grapefruit essential oil, and the pain relief composition may comprise 3.86 wt. % lavender essential oil and 2.32 wt. % grapefruit essential oil. As such, in certain embodiments, the pain relief composition may comprise fragrance at a weight percent from at least about any of the following: 0.025, 0.05, 0.1, 0.5, 1, 1.5, and 2 wt. % and/or at most about 40, 30, 20, 10, 9, 8, 7, 6, 5, and 4 wt. % (e.g., about 1-7 wt. %, about 2-5 wt. %, etc.).

### EXEMPLARY EMBODIMENTS

Having described various aspects and embodiments of the invention herein, further specific embodiments of the invention include those set forth in the following paragraphs.

In some example embodiments, a pain relief composition is provided. In general, the pain relief composition, according to certain embodiments, comprises moringa leaf, moringa oil, a gamma linolenic acid (GLA) source, and a capsaicin source.

In accordance with an example embodiment, the pain relief composition comprises from about 1 wt. % to about 40 wt. % moringa leaf. In some embodiments, the pain relief composition comprises from about 1 wt. % to about 40 wt. % moringa oil. In further embodiments, the pain relief composition comprises from about 0.5 wt. % to about 30 wt. % of the GLA source. In certain embodiments, the GLA source comprises at least one of evening primrose essential oil, borage seed oil, black currant seed oil, hemp seed oil, or any combination thereof. In some embodiments, the pain relief composition comprises from about 0.025 wt. % to about 20 wt. % of the capsaicin source. In certain embodiments, the capsaicin source comprises at least one of cayenne essential oil, black pepper essential oil, or any combination thereof.

According to an example embodiment, the pain relief composition further comprises fulvic acid and geranium essential oil. In some embodiments, the pain relief composition comprises from about 0.5 wt. % to about 30 wt. % fulvic acid. Moreover, in further embodiments, the pain relief composition comprises from about 0.025 wt. % to about 10 wt. % geranium essential oil.

According to an example embodiment, the pain relief composition further comprises at least one of cocoa butter, shea butter, coconut oil, olive oil, avocado oil, or any combination thereof. In some embodiments, the pain relief composition comprises from about 1 wt. % to about 60 wt. % cocoa butter. In further embodiments, the pain relief composition comprises from about 20 wt. % to about 80 wt. % shea butter. In certain embodiments, the pain relief composition comprises from about 1 wt. % to about 60 wt. % coconut oil. In some embodiments, the pain relief composition further comprises a fragrance. In such embodiments, the fragrance comprises at least one essential oil. In certain embodiments, the pain relief composition comprises from about 0.025 wt. % to about 40 wt. % fragrance. In further embodiments, the pain relief composition comprises a cream, a lotion, or an oil.

In accordance with an example embodiment, the pain relief composition comprises moringa leaf, moringa oil, evening primrose essential oil, cayenne essential oil, fulvic acid, geranium essential oil, and at least one of cocoa butter, shea butter, coconut oil, olive oil, avocado oil, or any combination thereof. In some embodiments, the pain relief composition comprises a cream, a lotion, or an oil. In certain embodiments, the pain relief composition comprises a cream, and the cream comprises from about 5 wt. % to about 7 wt. % moringa leaf, from about 5 wt. % to about 7 wt. % moringa oil, from about 2 wt. % to about 4 wt. % evening primrose essential oil, from about 0.6 wt. % to about 0.8 wt. % cayenne essential oil, from about 2 wt. % to about 4 wt. % fulvic acid, from about 0.2 wt. % to about 0.4 wt. % geranium essential oil, from about 8 wt. % to about 10 wt. % cocoa butter, from about 54 wt. % to about 56 wt. % shea butter, and from about 8 wt. % to about 10 wt. % coconut oil.

These and other modifications and variations to the present invention may be practiced by those of ordinary skill in the art without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

That which is claimed:

1. A pain relief composition comprising:
moringa leaf;
moringa oil;
a gamma linolenic acid (GLA) source; and
a capsaicin source.

2. The pain relief composition according to claim 1, wherein the pain relief composition comprises from about 1 wt. % to about 40 wt. % moringa leaf.

3. The pain relief composition according to claim 1, wherein the pain relief composition comprises from about 1 wt. % to about 40 wt. % moringa oil.

4. The pain relief composition according to claim 1, wherein the pain relief composition comprises from about 0.5 wt. % to about 30 wt. % of the GLA source.

5. The pain relief composition according to claim 1, wherein the pain relief composition comprises from about 0.025 wt. % to about 20 wt. % of the capsaicin source.

6. The pain relief composition according to claim 1, wherein the GLA source comprises at least one of evening primrose essential oil, borage seed oil, black currant seed oil, hemp seed oil, or any combination thereof.

7. The pain relief composition according to claim 1, wherein the capsaicin source comprises at least one of cayenne essential oil, black pepper essential oil, or any combination thereof.

8. The pain relief composition according to claim 1, further comprising:
fulvic acid; and
geranium essential oil.

9. The pain relief composition according to claim 8, wherein the pain relief composition comprises from about 0.5 wt. % to about 30 wt. % fulvic acid.

10. The pain relief composition according to claim 8, wherein the pain relief composition comprises from about 0.025 wt. % to about 10 wt. % geranium essential oil.

11. The pain relief composition according to claim 1, further comprising at least one of cocoa butter, shea butter, coconut oil, olive oil, avocado oil, or any combination thereof.

12. The pain relief composition according to claim 11, wherein the pain relief composition comprises from about 1 wt. % to about 60 wt. % cocoa butter.

13. The pain relief composition according to claim 11, wherein the pain relief composition comprises from about 20 wt. % to about 80 wt. % shea butter.

14. The pain relief composition according to claim 11, wherein the pain relief composition comprises from about 1 wt. % to about 60 wt. % coconut oil.

15. The pain relief composition according to claim 1, further comprising a fragrance, said fragrance comprising at least one essential oil.

16. The pain relief composition according to claim 15, wherein the pain relief composition comprises from about 0.025 wt. % to about 40 wt. % fragrance.

17. The pain relief composition according to claim 1, wherein the pain relief composition comprises a cream, a lotion, or an oil.

18. A pain relief composition comprising:
moringa leaf;
moringa oil;
evening primrose essential oil;
cayenne essential oil;
fulvic acid;
geranium essential oil; and
at least one of cocoa butter, shea butter, coconut oil, olive oil, avocado oil, or any combination thereof.

19. The pain relief composition according to claim 18, wherein the pain relief composition comprises a cream, a lotion, or an oil.

20. The pain relief composition according to claim 19, wherein the pain relief composition comprises a cream, and the cream comprises:
from about 5 wt. % to about 7 wt. % moringa leaf;
from about 5 wt. % to about 7 wt. % moringa oil;
from about 2 wt. % to about 4 wt. % evening primrose essential oil;
from about 0.6 wt. % to about 0.8 wt. % cayenne essential oil;
from about 2 wt. % to about 4 wt. % fulvic acid;
from about 0.2 wt. % to about 0.4 wt. % geranium essential oil;
from about 8 wt. % to about 10 wt. % cocoa butter;
from about 54 wt. % to about 56 wt. % shea butter; and
from about 8 wt. % to about 10 wt. % coconut oil.
